(19) United States
(12) Patent Application Publication
(10) Pub. No.: US 2025/0340823 A1
(43) Pub. Date: Nov. 6, 2025

(54) METHODS FOR RECOVERING CELL PRODUCTS FROM CELLULAR FACTORIES

(71) Applicant: BioSystOmics, Inc., Houston, TX (US)

(72) Inventor: Naveed Aslam, Bellaire, TX (US)

(73) Assignee: BioSystOmics, Inc., Houston, TX (US)

(21) Appl. No.: 19/267,754

(22) Filed: Jul. 14, 2025

Related U.S. Application Data
(63) Continuation-in-part of application No. 16/974,427, filed on Feb. 1, 2021.

(51) Int. Cl. C12M 1/00

(52) U.S. Cl. CPC C12M 47/06

(57) ABSTRACT

The instant disclosure is directed to a system for recovering intracellular products from cell factories. The system includes a GN₂ pressurized container and conduit positioned within and vertically traversing the container. A conduit inlet is positioned proximate to a bottom portion of the container. A conduit outlet is positioned within the container. The inlet receives liquid N₂ and cell factories that combine to form a cryogenic slurry. The cell factories produce an intracellular product. As the cryogenic slurry traverses through the conduit from the inlet to the outlet, the LN₂ is absorbed into cell walls the cell factories. When the cryogenic slurry is released at the outlet, the cell factories experience an increase in pressure caused by the GN₂ of the container, rupture, and thereby release the intracellular product. The GN₂ is recycled back into the container and the intracellular product is captured.

### CROSS-REFERENCE TO RELATED APPLICATIONS

This application is a continuation-in-part U.S. patent application Ser. No. 16/974,427, filed Feb. 21, 2021, the contents of which are incorporated by reference herein in their entirety.

### TECHNICAL FIELD

### Background

The present disclosure relates to the recovery of cell products from heterologous cellular expression systems. The following discussion is provided solely to assist the understanding of the reader and does not constitute an admission that any of the information discussed, or references cited constitute prior art to the present invention.

Biopharmaceuticals offer significant therapeutic value and have garnered considerable interest. However, medicinal proteins are typically difficult to extract in large quantities from natural biological sources, such as human blood or helminth secretions. Instead, most biopharmaceutical proteins are produced using recombinant DNA technology, which involves inserting a gene of interest into a host organism capable of being cultured to express the target protein. Heterologous expression systems serve as experimental platforms to produce genes or proteins in a host organism different from the gene's organism of origin. Suitable host organisms include plant cells, mammalian cells, yeast cells, bacterial cells, and algal cell systems.

Several heterologous expression systems are currently employed to produce biopharmaceuticals. For instance, most approved biopharmaceuticals for human use are generated using Escherichia coli, the yeast Saccharomyces cerevisiae, or mammalian cell lines such as Chinese Hamster Ovary (CHO) cells or murine myeloma (SP2/0) cells. Despite the large-scale production capabilities of these systems, careful consideration is essential when selecting an expression system for a specific application to ensure optimal performance and product quality.

For example, while Escherichia coli offers cost-effective biopharmaceutical production, prokaryotes like E. coli cannot synthesize complex proteins and often form large protein aggregates. Proteins expressed in E. coli typically require refolding to achieve their active state and lack the ability to perform complex post-translational modifications, such as N-glycosylation. Similarly, while yeast cells are generally inexpensive and straightforward to culture and can produce glycoproteins, they tend to generate mannose-enriched N-glycans and exhibit hyper-glycosylation. These mannose-enriched proteins may have a reduced half-life in the bloodstream due to rapid clearance, making them less suitable for pharmaceutical glycoproteins.

Human biopharmaceutical proteins can also be produced using plant cell cultures, which offer several advantages over mammalian cell cultures. These include cost-effectiveness and the absence of mammalian-derived components in the production process. Additionally, plant cell systems allow precise control overgrowth conditions, ensuring high batch-to-batch reproducibility and compliance with current Good Manufacturing Practice (CGMP). A key benefit of plant systems is their significantly lower production costs. Estimates suggest that producing recombinant proteins in plants can be 10-50 times less expensive than in Escherichia coli and up to 1,000 times cheaper than in Chinese Hamster Ovary (CHO) cell systems.

Plants provide a cost-effective platform for biopharmaceutical production. Utilizing existing infrastructure for crop cultivation, processing, and storage minimizes the capital investment needed for commercial production. Key advantages of plant cell systems for biopharmaceuticals include the ability to assemble complex multimeric proteins like antibodies, the capacity to perform post-translational modifications similar to those in mammalian cells, reduced operational costs, and straightforward scalability.

Plant cell cultures, while valuable, present several challenges. First, plant-based expression systems often require extensive downstream processing, with approximately 80-90% of the costs associated with plant-derived biopharmaceuticals attributed to these processes. Second, the final yield of recombinant antibodies from plant cells is typically lower due to uncontrolled proteolytic degradation. Third, plant-based proteins exhibit distinct glycosylation patterns that may differ from those in other systems, potentially affecting their functionality or immunogenicity.

### SUMMARY

Aspects of the present disclosure are illustrated by way of example and are not limited by the accompanying figures for which like references indicate like elements.

Cellular disruption is a method in which the outer boundary or cell membrane is broken down or destroyed in order to release intracellular materials such as DNA, RNA, protein or organelles from a cell factory. Cell disruption is an important unit operation for recovery of biopharmaceutical products from heterologous expression systems. It can be also a key step in the molecular diagnostics of pathogens, immunoassays for point of care diagnostics, down streaming processes such as protein purification for studying protein function and structure, cancer diagnostics, drug screening, mRNA transcriptome determination and analysis of the composition of specific proteins, lipids, and nucleic acids individually or as complexes.

Based on the application, cell lysis can be classified as complete or partial. Partial cell lysis is performed in methods like patch clamping, which is used for drug testing and studying intracellular ionic currents. In this technique, a glass micropipette is inserted into the cell, rupturing the cell membrane only partially and recovering the key target. Complete cell lysis is the full disintegration of cell membrane and is usually required for obtaining higher yield of biopharmaceutical products from expression systems. There are several cell disruption methods such as mechanical homogenizers, sonication, enzymatic, chemical, grinding, and osmotic shock are currently employed at industrial scale to disrupt the cellular systems.

However, all these methods are characterized by lower yield, higher cost, complex processing, loss of product, and longer cycle time. The instant disclosure is directed to a system for recovering intracellular products from cell factories. The system includes a container and conduit positioned within and vertically traversing the container. The container is pressurized by gaseous nitrogen (GN₂). The conduit has an inlet and outlet. The inlet is positioned proximate to a bottom portion of the container. The outlet is positioned within the container and proximate to a top portion of the container.

The inlet concurrently receives liquid N₂ (LN₂) and cell factories that combine therein to form a cryogenic slurry. The cell factories are configured to produce an intracellular product. As the cryogenic slurry traverses through the conduit from the inlet to the outlet, the LN₂ is absorbed into cell walls of at least a portion of the cell factories. When the cryogenic slurry is released at the outlet, the cryogenic slurry becomes a disrupted cryogenic slurry; and the cell factories experience an increase in pressure caused by the GN₂ of the container, rupture, and thereby release the intracellular product.

The GN₂ is captured and recycled back into the container. The disrupted cryogenic slurry travels towards the bottom portion and the intracellular product is captured. The unruptured cell factories are captured and reintroduced into the cryogenic slurry. The conduit includes a plurality of channels. As the cryogenic slurry traverses the conduit, a portion of the LN₂ converts to GN₂ and is released into the container via the plurality of channels. The container and/or the conduit has a pipe structure and/or tube structure. Inside the container, the disrupted cryogenic slurry separates into two or more of an intracellular products phase, biomass phase, and cellular factory phase. The cellular factory phase is captured and reintroduced into the cryogenic slurry.

The conduit includes one or more of a serpentine structure and a coiled structure. The container receives GN₂ at 5-55 atm. The cell factories include one or more of bacterial cells, microalgae cells, plants cells, mammalian cells, and insect cells. When the cryogenic slurry is released at the outlet, the cryogenic slurry is aerosolized. The container receives heated GN₂ having a temperature of 40-75° C. The heated GN₂ circulates within the container and thereby dries the intracellular product.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the Background.

### BRIEF DESCRIPTION OF THE DRAWINGS

FIG. 1 depicts a cross-section view of a Type I plant cell, in accordance with some embodiments.

FIG. 2 depicts a cross-section view of a Type II plant cell, in accordance with other embodiments.

FIG. 3 depicts a cross-section view of a Type Ill plant cell, in accordance with certain embodiments.

FIG. 4 depicts a diagram of a Type IV plant cell, in accordance with yet still other embodiments.

FIG. 5 depicts a diagram of a spray nozzle dispensing liquid nitrogen (LN₂) onto a mammalian cell to aid in cell disintegration, in accordance with some embodiments.

FIG. 6 depicts a diagram of pressurization and depressurization cycles of a mammalian cell using inert nitrogen or argon to accomplish cell disintegration, in accordance with certain embodiments.

FIG. 7 depicts a diagram of a modular, simpler, universal, tunable, lower-cost, higher yield and continuous process for disruption of plant, mammalian, algal, yeast, bacterial and insect cells through LN₂ spray pretreatment followed by lower pressure disintegration by pressurization/depressurization cycle with inert gas such as nitrogen or argon, in accordance with yet still other embodiments.

FIG. 8 depicts a diagram of LN₂-GAN system for insulin production from yeast cells, in accordance with other embodiments.

FIG. 9 depicts a cross-section view of an intracellular product recovery system, in accordance with some embodiments.

FIG. 10 depicts a close up of a section of the intracellular product recovery system of FIG. 9, in accordance with yet still other embodiments.

FIG. 11 depicts a cross-section view of an intracellular product recovery system, in accordance with other embodiments.

### DETAILED DESCRIPTION

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Certain terminology may be employed in the following description for convenience rather than for any limiting purpose. For example, the terms “forward” and “rearward,” “front” and “rear,” “right” and “left,” “upper” and “lower,” and “top” and “bottom” designate directions in the drawings to which reference is made, with the terms “inward,” “inner,” “interior,” or “inboard” and “outward,” “outer,” “exterior,” or “outboard” referring, respectively, to directions toward and away from the center of the referenced element, the terms “radial” or “horizontal” and “axial” or “vertical” referring, respectively, to directions or planes which are perpendicular, in the case of radial or horizontal, or parallel, in the case of axial or vertical, to the longitudinal central axis of the referenced element, the terms “proximate” and “distal” referring, respectively, to positions or locations that are close or away from a point of reference, and the terms “downstream” and “upstream” referring, respectively, to directions in and opposite that of fluid flow. Terminology of similar import other than the words specifically mentioned above likewise is to be considered as being used for purposes of convenience rather than in any limiting sense.

In the figures, elements having an alphanumeric designation may be referenced herein collectively or in the alternative, as will be apparent from context, by the numeric portion of the designation only. Further, the constituent parts of various elements in the figures may be designated with separate reference numerals which shall be understood to refer to that constituent part of the element and not the element as a whole. General references, along with references to spaces, surfaces, dimensions, and extents, may be designated with arrows.

Angles may be designated as “included” as measured relative to surfaces or axes of an element and as defining a space bounded internally within such element therebetween, or otherwise without such designation as being measured relative to surfaces or axes of an element and as defining a space bounded externally by or outside of such element therebetween. Generally, the measures of the angles stated are as determined relative to a common axis, which axis may be transposed in the figures for purposes of convenience in projecting the vertex of an angle defined between the axis and a surface which otherwise does not extend to the axis. The term “axis” may refer to a line or to a transverse plane through such line as will be apparent from context.

Biopharmaceuticals hold immense therapeutic value, yet extracting medicinal proteins from natural sources like human blood or helminth secretions is often impractical due to low yields. Recombinant DNA technology addresses this by inserting a gene of interest into a host organism for large-scale protein production. Heterologous expression systems, which produce genes or proteins in a host different from their origin, utilize organisms such as bacterial, yeast, mammalian, plant, or algal cells to achieve this.

Commonly used systems include Escherichia coli, Saccharomyces cerevisiae (yeast), and mammalian cell lines like Chinese Hamster Ovary (CHO) or murine myeloma (SP2/0) cells, which produce most approved biopharmaceuticals. Selecting the appropriate system requires careful evaluation to ensure optimal performance and product quality, as each has unique strengths and limitations.

For instance, E. coli is cost-effective but struggles with complex protein synthesis, often forming aggregates that require refolding to become active. It also lacks the ability to perform complex post-translational modifications like N-glycosylation. Yeast cells, while affordable and capable of producing glycoproteins, tend to generate mannose-enriched N-glycans, which may lead to rapid clearance in the bloodstream, reducing their suitability for pharmaceutical glycoproteins.

Plant cell cultures offer a compelling alternative, providing cost-effectiveness and eliminating mammalian-derived components, thus enhancing safety. They allow precise control overgrowth conditions, ensuring high batch-to-batch reproducibility and compliance with current Good Manufacturing Practice (cGMP). Production costs in plants are significantly lower, estimated to be 10-50 times less than in E. coli and up to 1,000 times less than in CHO systems. By leveraging existing agricultural infrastructure for cultivation, processing, and storage, plants further reduce capital investment. They excel at assembling complex proteins like antibodies, performing post-translational modifications akin to mammalian cells, and offering scalability at reduced costs.

However, plant-based systems face challenges, including extensive downstream processing, which accounts for 80-90% of costs, lower yields due to uncontrolled proteolytic degradation, and distinct glycosylation patterns that may impact protein functionality or immunogenicity.

Transgenic sources, particularly plants, provide significant cost advantages over bioreactor-based methods. Producing recombinant drugs in transgenic plants costs an estimated 10-20% of fermentation-based methods, with monoclonal antibody production in mammalian cell cultures ranging from $140/g to $450/g (20-40% for protein production, the rest for recovery and purification). In contrast, transgenic crops can produce drugs at $9/g to $15/g. Plants also avoid human pathogens, reducing the need for costly pathogen-clearance steps required in mammalian or animal-derived systems. Tobacco, among other plants like carrot, tomato, and maize, is a promising candidate due to minimal regulatory and safety concerns.

For insulin production, where global demand is projected to reach 1,600 kg/year by 2025 due to rising diabetes rates, yeast-based systems like Saccharomyces cerevisiae offer advantages over E. coli and mammalian cells. Yeast grows rapidly, is easy to manipulate, and produces insulin similar to mammalian cells. However, its tough cell walls require intensive disruption. The proposed LN₂ and GN₂ (or argon) treatment softens yeast cell walls, enabling gentler extraction at lower pressures (100-500 psi), improving yield and efficiency. This approach is scalable, cost-effective, and maintains insulin quality.

Similarly, microalgae cell wall disruption for biodiesel production benefits from LN₂ pre-treatment followed by a low-pressure GN₂ process, reducing energy input and enhancing lipid recovery. These innovations underscore the potential of transgenic and yeast-based systems to meet growing biopharmaceutical demands efficiently and economically.

The proposed invention introduces a multifunctional device designed for process intensification, performing multiple steps-LN₂ pre-treatment, cell disruption in pressurized GN₂, separation of intracellular products, drying, and recycling of undisrupted cells-in a single, energy-efficient unit. This continuous process reduces labor and energy costs while improving productivity compared to batch methods.

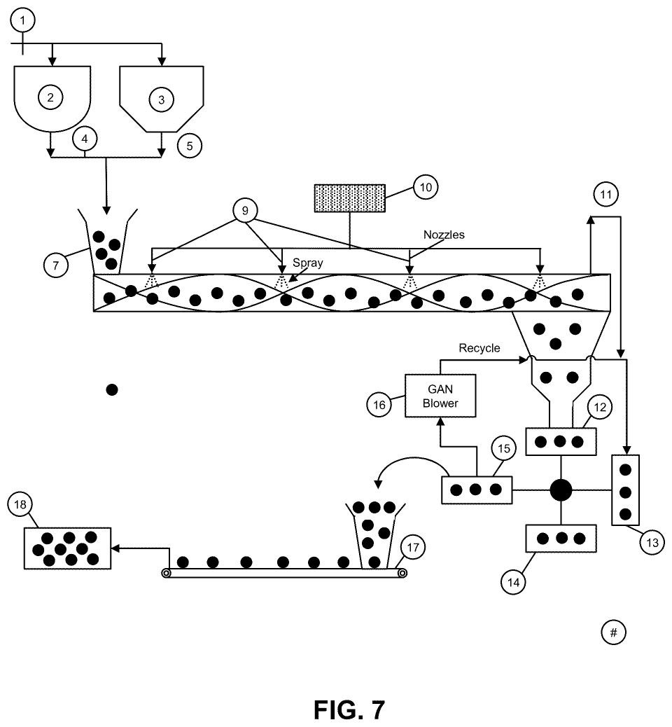

Disintegration of cellular factories into useful products like insulin, monoclonal antibodies (mAbs), lipids, DNA, carotenoids, oils and proteins can be accomplished as described in the process of FIG. 7. The LN₂-GAN based process described therein uses expression systems to generate intracellular products in a high yield and continuous manner to lower costs and reduce cycle times.

Recombinant proteins can be produced in plant systems through three primary strategies: cell cultures, plant tissue-based systems, and transgenic plants. Utilizing plants for biopharmaceutical production offers the potential to enhance yields and significantly reduce costs. Plant-based cell factories provide several advantages over animal-derived systems, including improved safety, lower production costs, higher yields, increased throughput, greater stability, and enhanced resilience to minor process variations.

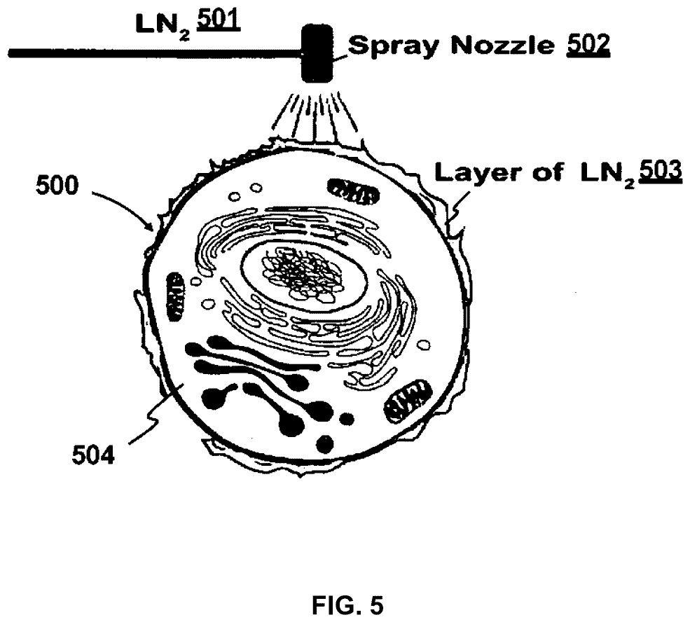

However, current downstream processing methods for plant cell factories often result in reduced recombinant protein yields due to inherent degradation processes within the plant cells. The pre-treatment method proposed, involving liquid nitrogen (LN₂) as illustrated in FIG. 5, can mitigate these degradation events, thereby preserving higher concentrations of target proteins in engineered cells. This approach has the potential to significantly enhance the yield of recombinant proteins from plant cell factories.

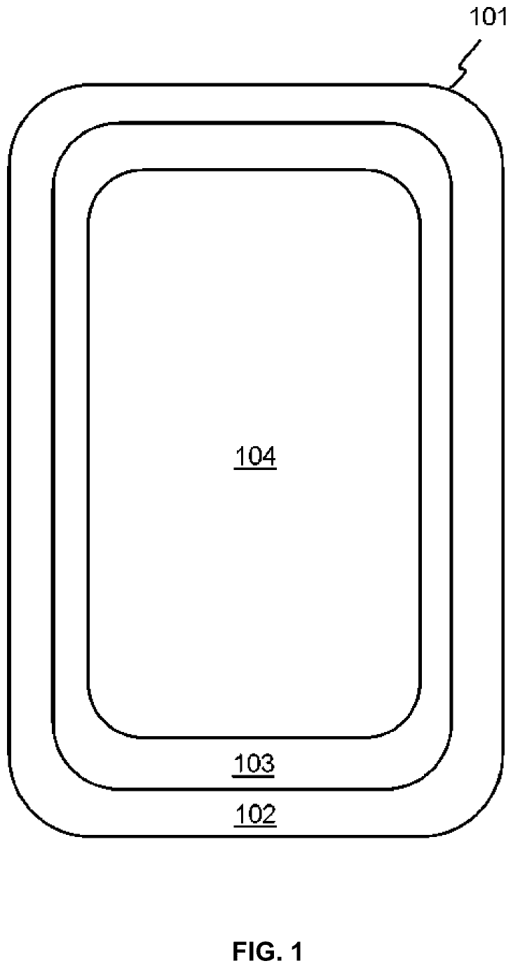

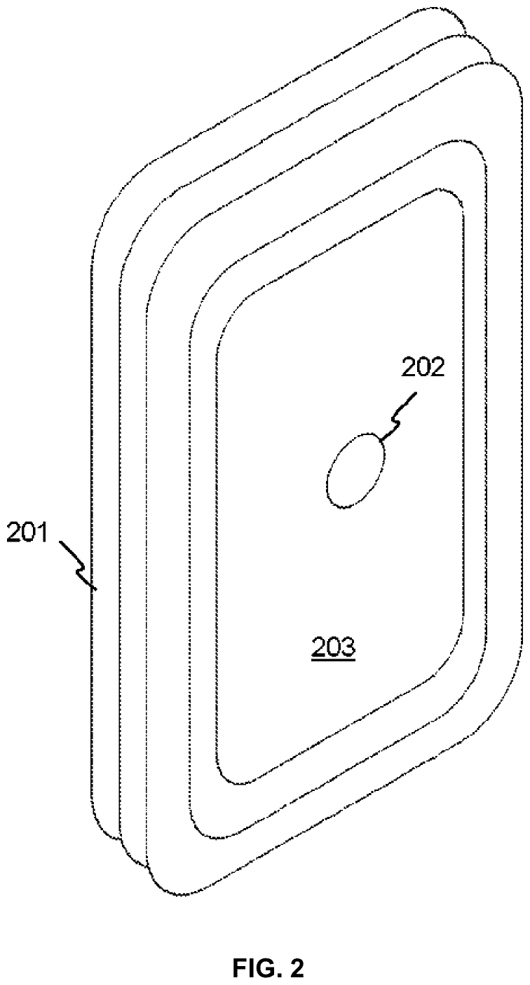

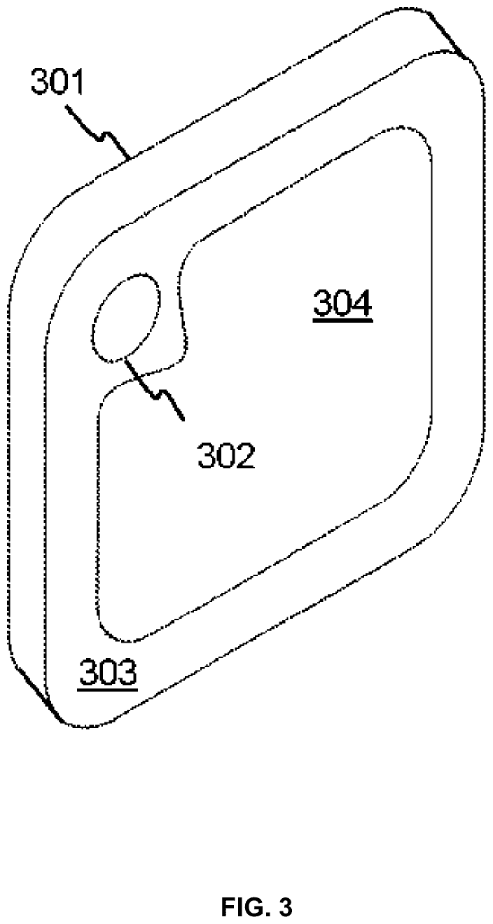

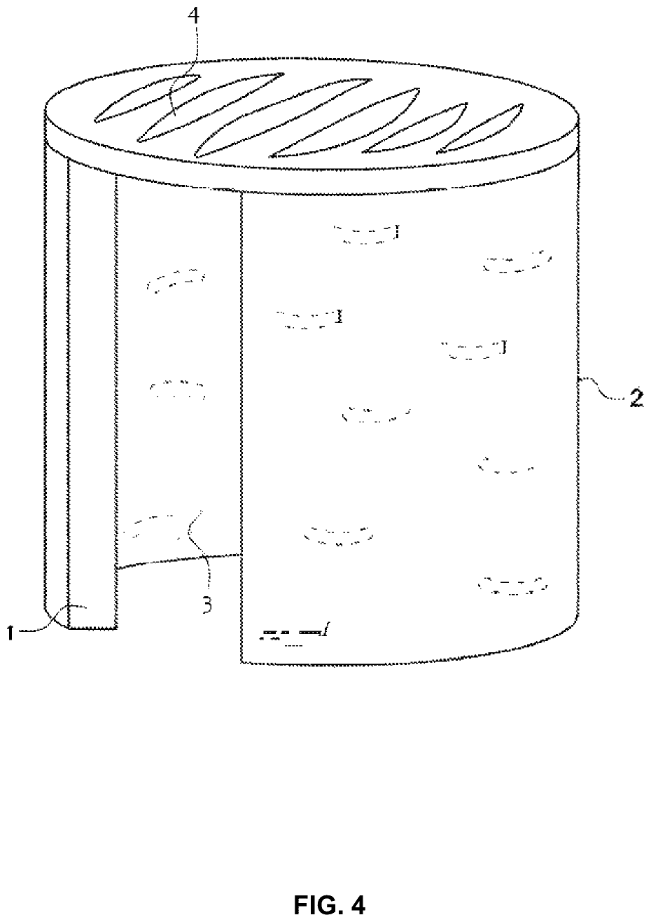

FIGS. 1-4 depict the cellular structure of several types of plant cells. As shown, the outer cell boundary of plant cell factories is an elaborate extracellular matrix that encloses each cell in the plant. Each plant cell is surrounded by a tough extracellular matrix in the form of a cell wall composed of a network of cellulose microfibrils and cross-linking glycans embedded in a highly cross-linked matrix of pectin polysaccharides.

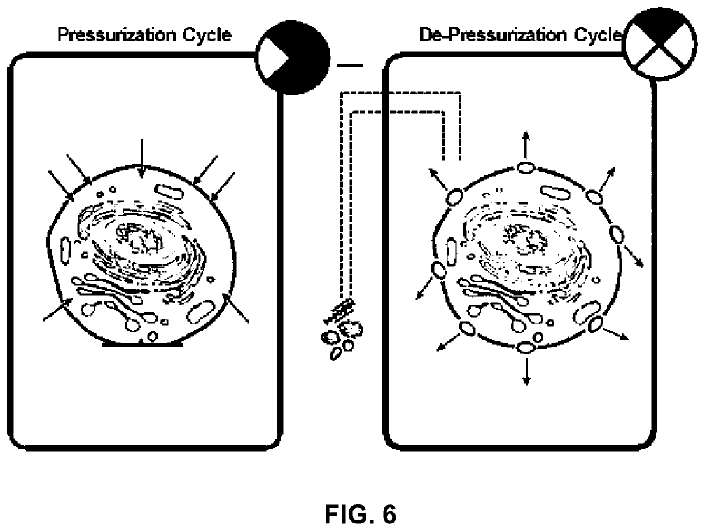

FIG. 6 illustrates a gentle and low-pressure batch process for disrupting mammalian or plant cells. In the process, cells are pre-treated, charged, as well as pressurized using gaseous nitrogen or argon to about 500 psi. The pressure is maintained for a predetermined time sufficient to promote dissolution of the gas into the cellular medium for proper equilibration. After equilibration, the vessel is depressurized. The depressurization profile is tunable. For example, depressurization can be abrupt or gradual depending on the cell type and product type.

Gas shear stresses induced by pressure reduction disrupt cellular structures can result in a more uniform and gentle disintegration of cellular mass. This approach minimizes product losses caused by aggregation and degradation, enabling the recovery of intact organelles with high yield. Unlike mechanical homogenization, which exposes cells to non-uniform stresses, leading to extensive grinding, destruction of cellular structures, protein aggregation, and potential yield losses, this system ensures all cells in a batch experience consistent stress, thereby enhancing overall efficiency and product recovery.

The instant disclosure seeks to provide biopharmaceutical processes that can be conducted in a batch configuration. Batch processing solutions known in the art typically suffer from high labor and operational costs as well as low yields and batch-to-batch yield variations. FIG. 7 illustrates a continuous process for cell disruption through LN₂-GAN treatments. Here, cells from fermentation batch (1) are transferred to storage vessels (2) and (3). Vessels (2) and (3) are configured to continuously supply fermented cells to the disruption process via lines (4) and (5), respectively.

Cells are transferred to hopper (7) via line (6). Hopper (7) gravity feeds fermented cells into spray chamber (8), which can include a ribbon screw or similar component. In the spray chamber (8), the LN₂ (10) is sprayed through nozzles (9) onto the fermented cells. The LN₂ can be aerosolized into a spray cloud, which acts as a pre-treatment step for cells like plant cells. As the fermented expression systems cells traverse the length of spray chamber (8) they are pre-treated with LN₂ (10). At the outlet of chamber (8), the pre-treated cells are gravity fed into an outlet hopper that supplies the pre-treated cells to vessel (12).

When the capacity of vessel (12) reaches one-third to one-half with cells, it moves to pressurization position (13) where the GAN supplied by blower (16) and recycled GAN (11) supplied by spray chamber (8) are used to pressurize vessel (13) to a predetermined value (e.g., determined by cell type and targeted intracellular product). Upon completion of the pressurization step, the vessel transitions to soaking position (14) where the vessel is allowed to equilibrate with GAN in the head space of the pressurized vessel and cellular materials located therein. Upon completion, the vessel is subsequently transitioned to depressurization position (15).

At depressurization position (15), the pressure from vessel is removed. Depressurization profile reflects specific cell type and targeted product. The GAN is recovered by blower (16) and recycled. The cellular material is charged on gravity hopper and transferred to conveyor belt (17), which communicates with polishing device (18). The batch process of FIG. 7 is a high yield and low-cost continuous cell disintegration process that can used for cellular systems having tough external walls, such as plant cells or algal cells.

Insulin production using E. coli expression system typically has certain disadvantages that include, but are not limited to, loss of plasmid and antibiotic properties, unsolicited gene expression, intracellular accumulation of heterologous proteins as inclusion bodies, improper protein folding, lack of post translational modification, protein-mediated metabolic burden and stress, endotoxin contamination, poor secretion, proteolytic digestion, and complexity in downstream processing. Given the foregoing, yeast expression system are arguably a more efficient expression system to produce insulin compared to E. coli and mammalian cell systems due to cost and quality of insulin yield.

For example, yeast-based systems grow rapidly and are amenable to various genetic manipulations. The recombinant insulin produced in yeast-based systems can be similar to that produced in mammalian cells. Since the 1980's, yeast cells such as Saccharomyces cerevisiae have been used to produce recombinant insulin in various batch sizes. The cell walls of yeast systems typically require extensive treatment (e.g., high pressure press) to rupture and then a high pressure or chemical-based system to extract the insulin from the ruptured cells.

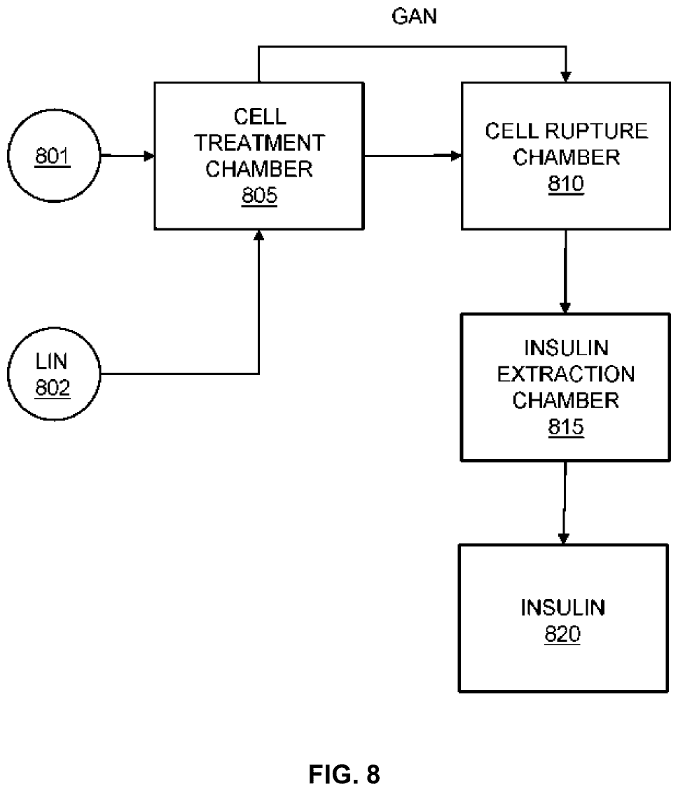

FIG. 8 depicts a block diagram of a LN₂-GN₂ system, generally 800, for insulin production from yeast cells, in accordance with other embodiments. In system 800, insulin bearing Saccharomyces cerevisiae (yeast) cell can be pre-treated with LN₂ to soften the cell walls. Treated yeast cells can be subjected to a pressure and depressurization cycle using GN₂, which generates shock wave stimulation to facilitate a gentler and more complete extraction of insulin. This method preserves the quality of the insulin extracted from the yeast cells. System 800 is an economical, scalable, faster and efficient process for producing insulin from yeast-based cell factories.

Phaffia rhodozyma is a type of yeast cell that can be used to produce carotenogenic pigments, which are typically used in aquaculture feed formulations, as well as the cosmetics, pharmaceuticals, and food industries. Carotenoids are natural pigments responsible for yellow, orange, and red in many foods such as fruits, vegetables, egg yolks, and fish (e.g., salmon and shellfish). However, production of carotenoids is typically hindered due to the cell wall resistance that reduces bioavailability. There exists a need to enhance the recovery of carotenoids from microorganisms for the food applications. Due to the concern about the use of chemical additives in foods, there is increasing interest in carotenoids naturally obtained by biotechnological processes. Therefore, the yeast Phaffia rhodozyma stands out as a natural source of carotenoids.

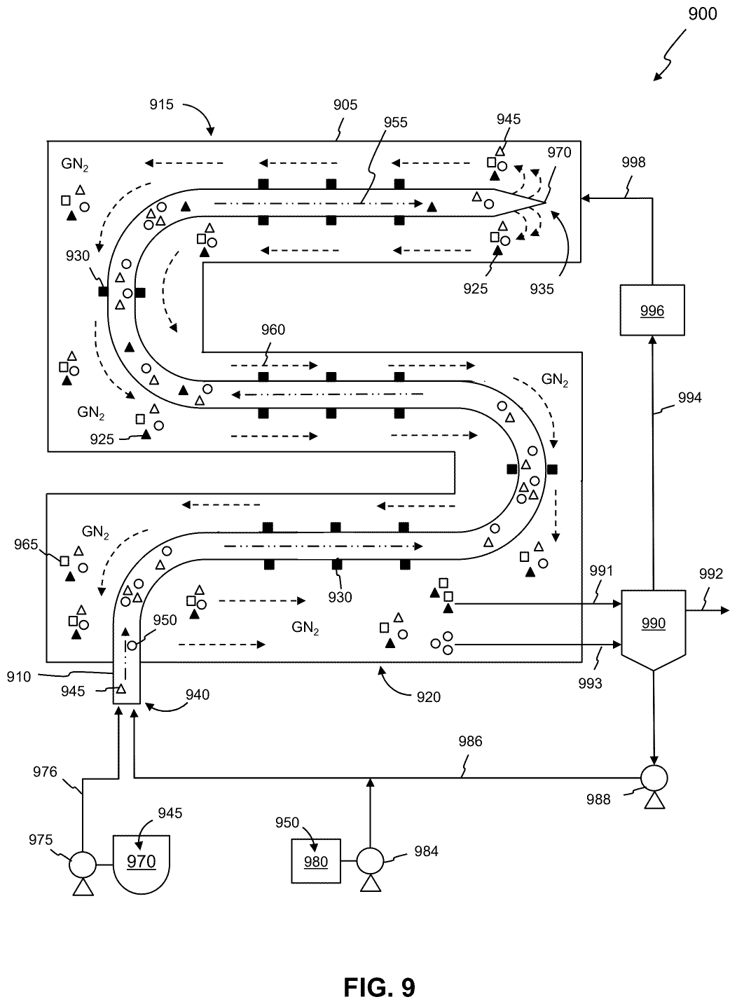

Turning now to FIGS. 9-11. Here, the instant disclosure seeks to provide a multifunctional device based on a process intensification design strategy. The multifunctional device is a single piece of hardware configured to accomplish a plurality of process steps while achieving improved energy efficiency and a smaller footprint compared to solutions known in the art or discussed above.

The multifunctional device can accomplish the following process steps: (a) LN₂ pre-treatment of cell factories; (b) cell disruption of cell factories in pressurized GN₂; (c) separation of intracellular products from disrupted cell biomass and undisrupted cell factories due to differences in terminal velocity of lighter intracellular products and heavier cellular biomass; (d) drying of intracellular products in heated GN₂ (e.g., 45-90° C.); and (e) complete disruption due to recycling of undisrupted cell factories. The multifunctional device is a single piece of hardware configured to perform steps (a) to (e) on a continuous basis, which can thereby reduce operating expenses related to labor and energy as well as improve overall productivity as compared to batch or semi batch processes.

FIG. 9 depicts a cross-section view of an intracellular product recovery system, generally 900, in accordance with some embodiments. System 900 is a system for recovering intracellular products from cell factories. The intracellular products are preferably biopharmaceutical products. Biopharmaceuticals can be composed of sugars, proteins, nucleic acids, or complex combinations of these substances, or may be living cells or tissues.

System 900 preferably includes container 905 and conduit 910 positioned therein. Conduit 910 is used to pretreat cell factories 950. Container 905 includes top portion 915 and bottom portion 920 positioned opposite thereto. Container 905 is pressurized and receives gaseous nitrogen GN₂. Container 905 is pressurized by the GN₂. In certain embodiments, conduit 910 vertically traverses container 905 from about bottom portion 920 to about top portion 915. Conduit 910 can be porous (i.e., includes a plurality of channels 930) and includes inlet 940 and outlet 935. Inlet 940 is positioned proximate to bottom portion 920 and outlet 935 is positioned proximate to top portion 915. Conduit 910 can have a serpentine structure and/or a coiled structure. Container 905 and/or conduit 910 can have a pipe structure and/or tube structure. LN₂ source 970 includes a vessel having LN₂ that is preferably fluidically coupled upstream to pump 975, which supplies LN₂ 945 to line 976. Cellular biomass source 980, which includes a vessel having cell factories 950, is fluidically coupled upstream to pump 984, which supplies cell factories 950 to line 986. Cell factories 950 are cells that produce intracellular product 965 and include a cell wall. Applicable cells can include bacterial cells, microalgae cells, plants cells, mammalian cells, insect cells, or a combination of two or more thereof. Inlet 940 concurrently receives LN₂ 945 via line 976 and cell factories 950 via line 986, which combine to form cryogenic slurry 955.

In some embodiments, as cryogenic slurry 955 traverses through conduit 910, a portion of LN₂ 945 converts to GN₂ 925, which can be released within container 905 via channels 930. Channels 930 can each have a diameter that allows only gas molecules to pass therethrough. In certain embodiments, channels 930 may comprises nozzles and/or holes. In preferred embodiments, cryogenic slurry 955 can traverse conduit 910 under a pressure that is slightly greater than atmospheric pressure (e.g., up to 5% greater).

During the pretreatment step, cryogenic slurry 955 traverses through conduit 910 and thereby allows the cell walls of cell factories 950 to be exposed to and absorb LN₂ 945, which increases their amenability to disintegration/disruption. During the cell disruption step, cryogenic slurry 955 is released into container 905 at outlet 935 and thereby becomes “disrupted cryogenic slurry 960.” Container 905 is preferably filled with pressured GN₂ 925 (e.g., a pressurized cloud of GN₂ 925). For example, disrupted cryogenic slurry 960 can be sprayed into container 905 via outlet 935, which can include at least one spray nozzle 970. When disrupted cryogenic slurry 960 enters container 905 (e.g., via spray nozzle 970), at least a portion of cell factories 950 experience an increase in pressure caused by GN₂ 925 that causes their cell walls to disintegrate (e.g., become disrupted). Upon disruption, cell factories 950 release intracellular product 965 stored therein. In some embodiments, cell disruption is not complete and disrupted cryogenic slurry 960 includes unruptured cell factories. Here, unruptured cell factories 950 can be captured and reintroduced (i.e., recycled) into cryogenic slurry 955. During the separation stage, disrupted cryogenic slurry 960 travels towards bottom portion 920 and intracellular product 965 is captured. For example, hopper 990 can be fluidically coupled downstream to container 905 via line 991 and line 993. Hopper 990 is configured to receive captured intracellular product 965 and GN₂ 925 via line 991.

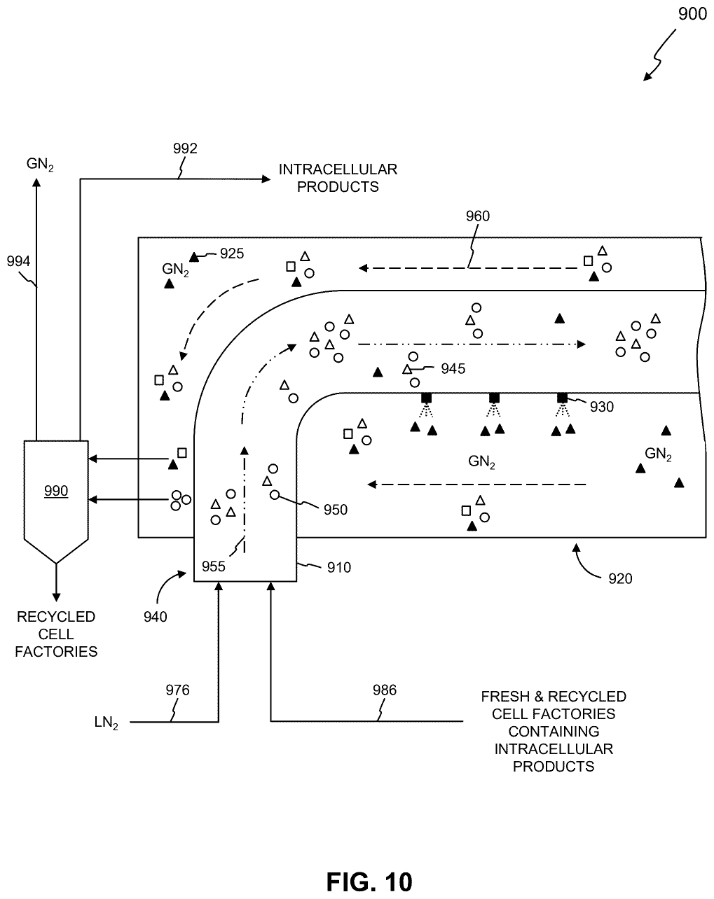

FIG. 10 depicts a close up of bottom portion 920 of system 900, according to yet still other embodiments. During the recycle stage, GN₂ 925 and cell factories 950 that were not disintegrated can be captured and recycled back into container 905. In certain embodiments, GN₂ 925 is captured and recycled back into container 905. For example, hopper 990 can be fluidically coupled downstream to container 905 via line 991 and line 993. Blower 996 can be fluidically coupled downstream to hopper 990 via line 994 and upstream to container 905 via line 998. Blower 996 can be configured to pressurize GN₂ 925 and transfer it to container 905 via line 998, which recycles GN₂ 925 and helps maintain the pressured environment of container 905. Hopper 990 may be configured to transfer captured intracellular product 965 to storage and/or further processing via line 992. In yet still other embodiments, cell factories 950 that were not disintegrated may be captured and sent to hopper 990 via line 993 and reintroduced to conduit 910 via line 986. Pump 988 can be fluidically coupled downstream to hopper 990 in a manner to transfer cell factories 950 from hopper 990 to line 986.

In other embodiments, inside container 905, disrupted cryogenic slurry 960 separates into phases subsequent to release at outlet 925 (e.g., via nozzle 935), which facilitate their capture. Applicable phases include an intracellular products phase, biomass phase, cellular factory phase, LN₂ phase, GN₂ phase, or a combination of two or more thereof. During the drying stage, heated GN₂ having a temperature between 40-75° C. can be introduced into container 905 to circulate therein and dry intracellular product 960. When dried, intracellular product 960 typically has a density difference compared to other components present in container 905, which allows dried intracellular product 960 to form a substantially distinct phase.

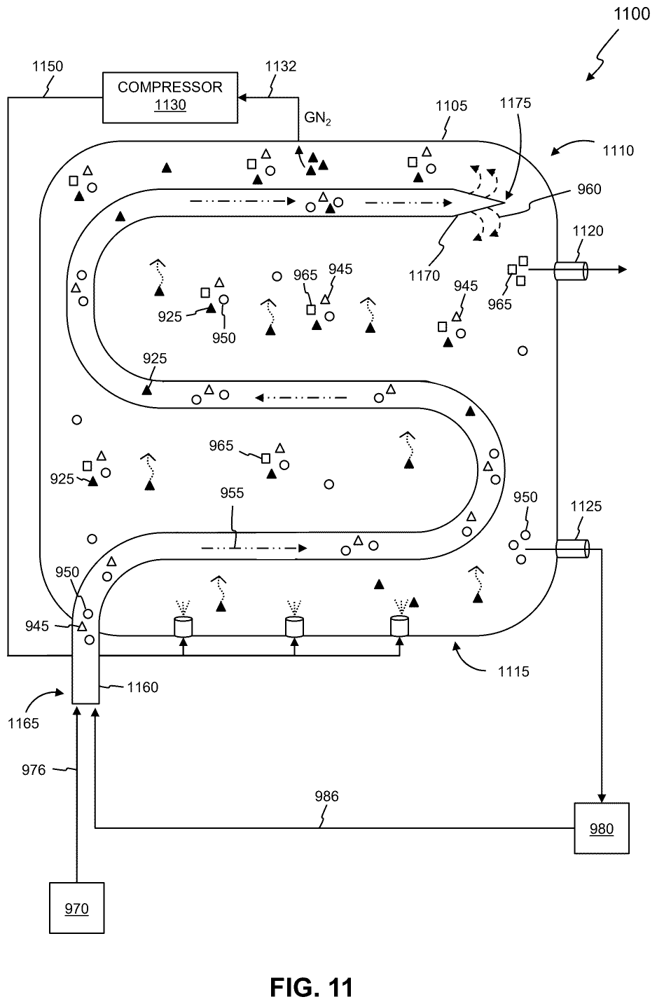

FIG. 11 depicts a cross-section view of an intracellular product recovery system, generally 1100, in accordance with other embodiments. In general, system 1100 can share one or more features, components, and/or processes with system 900. System 1100 preferably includes container 1105 and conduit 1160 positioned within container 1105. Conduit 1160 is preferably an elongated structure that transfers cryogenic slurry 955 from inlet 1165 to outlet 1170. At least one nozzle 1175 can be positioned proximate to outlet 1170. Cryogenic slurry 955 can be sprayed into container 1105 via nozzle 1175. In other words, nozzle 175 can be configured to aerosolize cryogenic slurry 955. Line 976 and line 986 transfer their contents to conduit 1160 via inlet 1165. As previously discussed in reference to FIGS. 9-10, line 976 fluidically couples LN₂ source 970 to inlet 1165 and line 986 fluidically couples cell biomass source 980 to inlet 1165. In other words, LN₂ source 970 and cell biomass source 980 are fluidically coupled upstream to inlet 1165 via line 976 and line 986, respectively.

For example, container 1105 can have an overall shape that substantially encloses conduit 1160 therein. Container 1105 can include one or more features, components, and/or processes of container 905. In preferred embodiments, container 1105 functions as an outer fluidized bed and conduit 910 functions as a pre-treatment space to achieve complete and high yielding recovery of intracellular products 965.

System 1100 is configured to capture and recycle GN₂ 925 in a unique manner compared to system 900. For example, in contrast to system 900, GN₂ 925 present in container 1105 is allowed to rise proximate to top portion 1110 and be transferred to compressor 1130 via line 1132. Compressor 1130 compresses captured GN₂ 925 to a predetermined pressure (e.g., 5-55 atm) and reintroduced to container 1105 via line 1150. In preferred embodiments, GN₂ 925 is reintroduced proximate to bottom portion 1115 as a fluidized medium that rises back towards top portion 1110 to be recaptured and thereby restart the recycling process.

Conduit 1160 is preferably not porous, which thereby causes GN₂ 925 generated within cryogenic slurry 955 to be released into container 1105 at outlet 1170, which is preferably positioned proximate to top portion 1110. As previously discussed, when cryogenic slurry 955 is released into container 905 at outlet 935 it is referred to as “disrupted cryogenic slurry 960.” Here, disrupted cryogenic slurry 960 at least includes LN₂ 945, GN₂ 925, cell factories 950, intracellular products 965, and/or biomass. As cell factories 950 are exposed to higher pressure within container 1105 they undergo the disruption process whereby their cell wall ruptures and release intracellular products 965.

Given that intracellular products 965 weight less than cell factories 950, their terminal falling velocity is lower than that of cell factories 950, which allow intracellular products 965 to be collected at outlet 1120 of container 1105. The heavier undisrupted cell factories 950 will continue their downward journey in the rising GN₂ environment, which may result in additional disruption and disintegration. Cell factories 950 that remain undisrupted are captured and transferred via outlet 1125 to cellular biomass source 980 for subsequent recycling and mixing with fresh cell factories 950 to thereafter undergo another round of pretreatment and disruption/disintegration. In this manner, system 1100 can accomplish the complete disruption of cell factories and high yield of intracellular products at milder conditions and lower cost.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any two different values as the endpoints of a range or by taking two different range endpoints from specified ranges as the endpoints of an additional range. Such ranges are also within the scope of the described invention. Further, specification of a numerical range including values greater than one includes specific description of each integer value within that range.

Based on the foregoing, systems and methods have been disclosed in accordance with the instant disclosure. However, numerous modifications and substitutions can be made without deviating from the scope of the instant disclosure. Therefore, the instant disclosure has been disclosed by way of example and not limitation.

What is claimed is:

1. A system for recovering intracellular products from cell factories, comprising:
a container;
a conduit positioned within and vertically traversing the container;
wherein
the container is pressurized by gaseous nitrogen (GN₂);
the conduit comprises an inlet and an outlet, the inlet is positioned proximate to a bottom portion of the container, the outlet is positioned within the container and proximate to a top portion of the container;
the inlet concurrently receives liquid N₂ (LN₂) and cell factories that combine therein to form a cryogenic slurry, the cell factories produce an intracellular product;
as the cryogenic slurry traverses through the conduit from the inlet to the outlet, the LN₂ is absorbed into cell walls of at least a portion of the cell factories;
when the cryogenic slurry is released at the outlet,
the cryogenic slurry becomes a disrupted cryogenic slurry;
the cell factories experience an increase in pressure caused by the GN₂ of the container, rupture, and thereby release the intracellular product;
the GN₂ is captured and recycled back into the container; and
the disrupted cryogenic slurry travels towards the bottom portion and the intracellular product is captured.

2. The system of claim 1, wherein
the disrupted cryogenic slurry comprises unruptured cell factories; and
the unruptured cell factories are captured and reintroduced into the cryogenic slurry.

3. The system of claim 1, wherein
the conduit comprises a plurality of channels; and
as the cryogenic slurry traverses the conduit, a portion of the LN₂ converts to GN₂ and is released into the container via the plurality of channels.

4. The system of claim 1, wherein one or more of the container or the conduit comprises a pipe structure or a tube structure.

5. The system of claim 1, wherein
inside the container, the disrupted cryogenic slurry separates into two or more of an intracellular products phase, a biomass phase, and a cellular factory phase.

6. The system of claim 5, wherein
the cellular factory phase is captured and reintroduced into the cryogenic slurry.

7. The system of claim 1, wherein
the conduit comprises one or more of a serpentine structure and a coiled structure.

8. The system of claim 1, wherein
the container receives GN₂ at 5-55 atm.

9. The system of claim 1, wherein
the cell factories comprise one or more of bacterial cells, microalgae cells, plants cells, mammalian cells, and insect cells.

10. The system of claim 1, wherein
when the cryogenic slurry is released at the outlet, the cryogenic slurry is aerosolized.

11. The system of claim 1, wherein
the container receives heated GN₂ comprising a temperature of 40-75° C.; and
the heated GN₂ circulates within the container and thereby dries the intracellular product.

12. A system for recovering intracellular products from cell factories, comprising:
a container;
a conduit positioned within and vertically traversing the container;
wherein
the container is pressurized by gaseous nitrogen (GN₂);
the conduit comprises an inlet and an outlet, the inlet is positioned proximate to a bottom portion of the container, the outlet is positioned within the container and proximate to a top portion of the container;
the inlet concurrently receives liquid N₂ (LN₂) and cell factories that combine therein to form a cryogenic slurry, the cell factories produce an intracellular product;
as the cryogenic slurry traverses through the conduit from the inlet to the outlet, the LN₂ is absorbed into cell walls of at least a portion of the cell factories;
when the cryogenic slurry is released at the outlet,
the cryogenic slurry becomes a disrupted cryogenic slurry;
the cell factories experience an increase in pressure caused by the GN₂ of the container, rupture, and thereby release the intracellular product;
the GN₂ is captured and recycled back into the container; and
the disrupted cryogenic slurry travels towards the bottom portion and the intracellular product is captured;
the container receives heated GN₂ comprising a temperature of 40-75° C.; and
the heated GN₂ circulates within the container and thereby dries the intracellular product.

13. The system of claim 12, wherein
the disrupted cryogenic slurry comprises unruptured cell factories; and
the unruptured cell factories are captured and reintroduced into the cryogenic slurry.

14. The system of claim 13, wherein
the conduit comprises a plurality of channels; and
as the cryogenic slurry traverses the conduit, a portion of the LN₂ converts to GN₂ and is released into the container via the plurality of channels.

15. The system of claim 14, wherein one or more of the container or the conduit comprises a pipe structure or a tube structure.

16. The system of claim 15, wherein
inside the container, the disrupted cryogenic slurry separates into two or more of an intracellular products phase, a biomass phase, and a cellular factory phase.

17. The system of claim 16, wherein
the cellular factory phase is captured and reintroduced into the cryogenic slurry.

18. The system of claim 17, wherein
the conduit comprises one or more of a serpentine structure and a coiled structure.

19. The system of claim 18, wherein one or more of
the container receives GN₂ at 5-55 atm; and
when the cryogenic slurry is released at the outlet, the cryogenic slurry is aerosolized.

20. The system of claim 19, wherein
the cell factories comprise one or more of bacterial cells, microalgae cells, plants cells, mammalian cells, and insect cells.
